SEQUENCE LISTING (6 sequences)

SEQ ID NO: 1 - DNA, 48 bp, Artificial Sequence
  Template
  misc_feature (19)..(30): n is Pyrimido[4,5-d]pyrimidine-2,4,5,7(1H,3H,6H,8H)-tetrone
cggtacctgcatgccgacnnnnnnnnnnnncagctagactagagctcg

SEQ ID NO: 2 - DNA, 18 bp, Artificial Sequence
  Primer
cgagctctagtctagctg

SEQ ID NO: 3 - DNA, 12 bp, Artificial Sequence
  Universal Template
catggatcctga

SEQ ID NO: 4 - DNA, 18 bp, Artificial Sequence
  Primer
cgagctctagtctagctg

SEQ ID NO: 5 - DNA, 18 bp, Artificial Sequence
  Primer
cggtacctgcatgccgac

SEQ ID NO: 6 - DNA, 12 bp, Artificial Sequence
  Template
catggatcctga

(19) United States
(12) Patent Application Publication
(10) Pub. No.: US 2018/0195099 A1
(43) Pub. Date: Jul. 12, 2018

(54) ENZYMATIC SYNTHESIS OF NUCLEIC ACID SEQUENCES

(71) Applicant: Thermo Fisher Scientific Geneart GmbH, Regensburg (DE)

(72) Inventors: Christian KRANZ, Regensburg (DE); Michael LISS, Regensburg (DE); Axel TREFZER, Tegernheim (DE)

(21) Appl. No.: 15/742,237

(22) PCT Filed: Jul. 7, 2016
(86) PCT No.: PCT/US16/41317

§ 371 Date: Jan. 5, 2018

Related U.S. Application Data
(60) Provisional application No. 62/189,439, filed on Jul. 7, 2015.

(51) Int. Cl. C12P 19/34

(52) U.S. Cl. CPC C12P 19/34

(57) ABSTRACT

The disclosure generally relates to compositions and methods for the enzymatic synthesis of nucleic acid sequences. In particular embodiments 3′ protected nucleotides are used in conjunction with a universal template to direct synthesis.

### CROSS-REFERENCE TO RELATED APPLICATIONS

This application is a 371 of International Application No. PCT/US2016/041317 filed Jul. 7, 2016, which application claims the benefit of U.S. Provisional Application No. 62/189,419 filed Jul. 7, 2015, the entire contents of which are hereby incorporated by reference in their entirety.

### REFERENCE TO SEQUENCE LISTING

The instant application contains a Sequence Listing which has been submitted electronically in ASCII format and is hereby incorporated by reference in its entirety. Said ASCII copy, created on Sep. 21, 2016, is named LT01066PCT_SL25.txt and is 1,554 bytes in size.

### FIELD OF THE INVENTION

The disclosure generally relates to methods and compositions for the enzymatic synthesis of nucleic acid molecules. In some aspects, the invention allows for the synthesis of nucleic acid molecules without the use of chemical steps and may involve the use of a universal template.

### BACKGROUND

Commercial oligonucleotide synthesis is an established technology that typically relies on the chemical addition of nucleotides using phosphoramidite chemistry. Using this approach, nucleotides are added to the 5′ terminus of the growing oligonucleotide chain in a process comprising four steps, deblocking, coupling, capping and oxidation. This process requires the use of chemical reagents that may be expensive and/or hazardous. Chemical synthesis is also error prone which may limit the length of oligonucleotide which can be produced. An aspect of the present invention is to provide a method of oligonucleotide synthesis that reduces or eliminates the need for chemical reaction steps and the associated reagents, has a low error rate and can produce longer oligonucleotides.

### SUMMARY OF THE INVENTION

The invention relates, in part, to compositions and methods for the enzymatic synthesis of nucleic acid molecules. In some aspects the sequence of the synthesized nucleic acid molecule is determined by the order of nucleotides added and not by the sequence of a template molecule. The invention relates in part to a single or double stranded nucleic acid template having a primer binding site at the 3′ end and a sequence of universal bases at the 5′ end. The single stranded nucleic acid template may be bound to a solid support at either its 3′ or 5′ end. A primer, complimentary to the primer binding site at the 3′ end, may then be added creating a duplex nucleic acid with an extended 5′ tail comprising universal bases. Synthesis of the nucleic acid occurs by the stepwise addition of 3′ protected nucleotides to the elongated primer using a polymerase. Each addition of a protected nucleotide is followed by a deprotection step to allow the addition of the next nucleotide in the sequence.

An exemplary method for the synthesis of an oligonucleotide may comprise a) providing on a solid support a single or double stranded template comprising a primer binding site at the 3′ end and a universal template at the 5′ end, b) adding a primer complimentary to the primer binding site, c) adding a polymerase and a 3′ protected nucleotide such that the protected nucleotide is added to the primer or the oligonucleotide extending from the primer, d) removing unreacted protected nucleotide and optionally polymerase and, e) removing the protective group from the 3′ protected nucleotide; and f) repeating steps (c)-(e) until synthesis of the oligonucleotide has been completed.

In some embodiments the solid support may be a surface such as a well, a tube or a resin which may be in a column or a bead. The surface may be smooth or porous. Solid supports may be synthetic or modified naturally occurring polymers, such as nitrocellulose, carbon, cellulose acetate, polyvinyl chloride, polyacrylamide, cross linked dextran, agarose, polyacrylate, polyethylene, polypropylene, poly (4-methylbutene), polystyrene, polymethacrylate, poly(ethylene terephthalate), nylon, poly(vinyl butyrate), polyvinylidene difluoride (PVDF) membrane, glass, controlled pore glass, magnetic controlled pore glass, magnetic or non-magnetic beads, ceramics, metals, and the like; either used by themselves or in conjunction with other materials.

In other embodiments the universal template may be comprised of inosine which is capable of forming a base pair with adenine, cytosine, guanine and thymine. In some embodiments, a base, other than inosine, that is able to transform its structure through keto-enol tautomerization and therefore able to base pair with all four naturally occurring nucleobases may be used to form a universal template. One example of such a universal base is Pyrimido[4,5-d]pyrimidine-2,4,5,7(1H,3H,6H,8H)-tetrone (PPT) available from Tokoyo Future Style Inc., Ibaraki, Japan.

In further embodiments, the polymerase used may be E. coli DNA polymerase, SEQUENASE 2.0™, T4 DNA polymerase or the Klenow fragment of DNA polymerase 1, T3, SP6 RNA polymerase, AMV, M-MLV, and/or Vent polymerase, as well as THERMOSEQUENASE™ (Amersham) or Taquenase™ (ScienTech, St Louis, Mo.) and THERMINATOR™ or Bst 2.0 DNA Polymerase (NEB, Ipswich, Mass.). DNA polymerase I, Klenow fragment. T4 DNA polymerase or a thermostable polymerase such as Taq polymerase.

In some embodiments the 3′ nucleotide may be protected using 3′-O-allyl, 3′-O-methoxymethyl, 3′-O-nitrobenzyl, 3′-O-azidomethylene, 3′-O-aminoalkoxyl or an aminoalkoxyl group such as 3′-ONH₂. In some embodiments the 3′-ONH₂ protecting group may be removed using a nitrite buffer at a pH of between 5 and 6.

Use of enzymes for nucleic acid synthesis instead of conventional phosphoramidite chemistry provides a number of advantages. The enzymatic approach does not require the use organic solvents reducing both the cost and environmental impact of the process. The enzymatic synthesis based invention described herein is applicable to high throughput applications such as ink jet based oligonucleotide synthesis (Blanchard A P, Kaiser R J, Hood L E. High-density oligonucleotide arrays. Biosensors Bioelectron. 1996; 11:687-690. doi: 10.1016/0956-5663(96)83302-1) or the plate- or chip-based synthesis system described in WO2016094512. Nucleic acids produced by the present invention could be used for gene synthesis, the production of gene arrays or gene chips or for diagnostic or therapeutic applications where the use of defined nucleic acid sequences is needed.

In many embodiments the invention may be automated. Automated systems are often driven by software which may perform repetitive tasks, especially when integrated with hardware designed for manipulation of components and reagent flows. Thus, according to various embodiments described herein, methods of synthesizing and assembling nucleic acids may be implemented on a computing system. Further, according to various embodiments described herein, processor-executable instructions for synthesizing and assembling nucleic acids are disclosed. Thus, in some aspects the invention includes non-transitory computer-readable storage media encoded with instructions, executable by a processor, for generating synthesized nucleic acid molecules, the instructions comprising instructions for:

a) adding to a solid support comprising a single stranded template comprising a primer binding site at the 3′ end and a universal template at the 5′ end a primer complimentary to the primer binding site,

b) adding a polymerase,

c) adding a 3′ protected nucleotide such that the protected nucleotide is added to the primer or the oligonucleotide extending from the primer,

d) removing unreacted protected nucleotide,

e) removing the protective group from the 3′ protected nucleotide; and

f) repeating steps (c)-(e) and optionally step (b) until synthesis of the oligonucleotide has been completed.

In alternate embodiments, step c) could comprise two or more 3′ protected nucleotides so that degenerate oligonucleotides could be produced. The mixture of 3′ protected nucleotides could be equimolar or of varying concentrations.

In other embodiments, step d) may further comprise removal of the polymerase.

In a further embodiment instructions may be provided for (g) combining the nucleic acid molecules generated in (f) to produce a pool;

(h) joining some or all of the nucleic acid molecules present in the pool formed in (g) to form a plurality of larger nucleic acid molecules;

(i) eliminating nucleic acid molecules which contain sequence errors from the plurality of larger nucleic acid molecules formed in (h) to produce an error corrected nucleic acid molecule pool.

In some embodiments, the instructions encoded on the non-transitory computer-readable storage media, executable by a processor, may include obtaining the sequence of the nucleotide to be synthesized from a user. In particular embodiments the user may enter the sequence using a keyboard or similar input device or the user may supply a digital file such as a FASTA file which contains the sequence to be synthesized.

### BRIEF DESCRIPTION OF THE FIGURES

FIG. 1 is a general description of one embodiment of the invention showing the process for adding a single nucleotide to a primer using a universal template.

FIG. 2 is a flow chart of an exemplary process for synthesis of error-minimized nucleic acid molecules.

FIG. 3 is a work flow chart of an exemplary process for synthesis of error-minimized nucleic acid molecules. Different strands of a double-stranded nucleic acid molecule are represented by thicker and thinner line. “MME” refers to mis-match endonuclease. Small circles represent sequence errors.

### DETAILED DESCRIPTION OF THE INVENTION

### Definitions

Nucleic Acid Molecule: As used herein the term “nucleic acid molecule” refers to a covalently linked sequence of nucleotides or bases (e.g., ribonucleotides for RNA and deoxyribonucleotides for DNA but also include DNA/RNA hydrids where the DNA is in separate strands or in the same strands) in which the 3′ position of the pentose of one nucleotide is joined by a phosphodiester linkage to the 5′ position of the pentose of the next nucleotide. Nucleic acid molecule may be single- or double-stranded or partially double-stranded. Nucleic acid molecule may appear in linear or circularized form in a supercoiled or relaxed formation with blunt or sticky ends and may contain “nicks”. Nucleic acid molecule may be composed of completely complementary single strands or of partially complementary single strands forming at least one mismatch of bases. Nucleic acid molecule may further comprise two self-complementary sequences that may form a double-stranded stem region, optionally separated at one end by a loop sequence. The two regions of nucleic acid molecule which comprise the double-stranded stem region are substantially complementary to each other, resulting in self-hybridization. However, the stem can include one or more mismatches, insertions or deletions.

Nucleic acid molecules may comprise chemically, enzymatically, or metabolically modified forms of nucleic acid molecules or combinations thereof. The invention provides, in part, compositions and combined methods relating to the enzymatic synthesis and assembly of nucleic acid molecules.

Nucleic acid molecule also refers to short nucleic acid molecules, often referred to as, for example, primers or probes. Primers are often referred to as single-stranded starter nucleic acid molecules for enzymatic assembly reactions whereas probes may be typically used to detect at least partially complementary nucleic acid molecules. A nucleic acid molecule has a “5′-terminus” and a “3′-terminus” because nucleic acid molecule phosphodiester linkages occur between the 5′ carbon and 3′ carbon of the pentose ring of the substituent mononucleotides. The end of a nucleic acid molecule at which a new linkage would be to a 5′ carbon is its 5′ terminal nucleotide. The end of a nucleic acid molecule at which a new linkage would be to a 3′ carbon is its 3′ terminal nucleotide. A terminal nucleotide or base, as used herein, is the nucleotide at the end position of the 3′- or 5′-terminus. A nucleic acid molecule sequence, even if internal to a larger nucleic acid molecule (e.g., a sequence region within a nucleic acid molecule), also can be said to have 5′- and 3′-ends.

Solid Support: As used herein, the term solid support refers to a porous or non-porous material on which polymers such as nucleic acid molecules can be synthesized and/or immobilized. As used herein “porous” means that the material contains pores which may be of non-uniform or uniform diameters (for example in the nm range). Porous materials include paper, synthetic filters etc. In such porous materials, the reaction may take place within the pores. The solid support can have any one of a number of shapes, such as pin, strip, plate, disk, rod, fiber, bends, cylindrical structure, planar surface, concave or convex surface or a capillary or column. The solid support can be a particle, including bead, microparticles, nanoparticles and the like. The solid support can be a non-bead type particle (e.g., a filament) of similar size. The support can have variable widths and sizes. For example, sizes of a bead (e.g., a magnetic bead) which may be used in the practice of the invention are described in WO2016094512. The support can be hydrophilic or capable of being rendered hydrophilic and includes inorganic powders such as silica, magnesium sulfate, and alumina; natural polymeric materials, particularly cellulosic materials and materials derived from cellulose, such as fiber containing papers such as filter paper, chromatographic paper or the like. The support can be immobilized at an addressable position of a carrier. The support can be loose (such as, e.g., a resin material or a bead in a well) or can be reversibly immobilized or linked to the carrier (e.g. by cleavable chemical bonds or magnetic forces etc.).

In some embodiments, solid support may be fragmentable. Solid supports may be synthetic or modified naturally occurring polymers, such as nitrocellulose, carbon, cellulose acetate, polyvinyl chloride, polyacrylamide, cross linked dextran, agarose, polyacrylate, polyethylene, polypropylene, poly (4-methylbutene), polystyrene, polymethacrylate, poly(ethylene terephthalate), nylon, poly(vinyl butyrate), polyvinylidene difluoride (PVDF) membrane, glass, controlled pore glass (CPG), magnetic controlled pore glass, magnetic or non-magnetic beads, ceramics, metals, and the like; either used by themselves or in conjunction with other materials.

### Overview

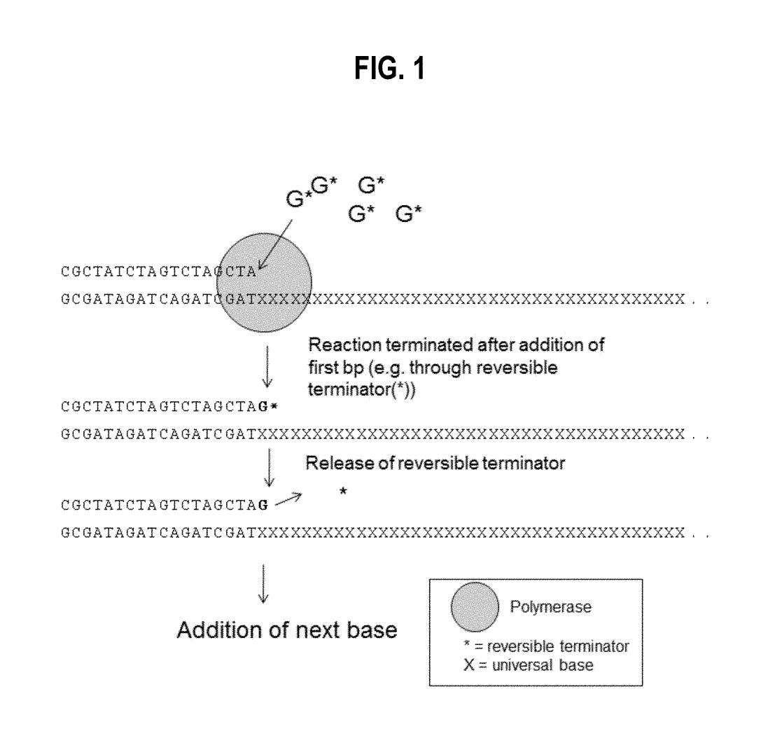

The invention relates in part to methods and compositions for the synthesis of nucleic acid molecules. In conventional enzymatic nucleic acid synthesis the sequence of the synthesized molecule is determined by the sequence of the template. In embodiments of the present invention, a universal template is used and the sequence of the synthesized molecule is determined by the order in which individual bases are attached to the growing nucleotide chain. While the invention has various aspects, one exemplary version is outlined in FIG. 1. In this embodiment, a single stranded universal template is used to direct synthesis of the nucleic acid molecule. The universal template comprises a primer binding site at its 3′ end. The primer binding site may be from 10 to 25, 10 to 20, 10 to 15, 15 to 25, or 15-20 nucleotides in length. In some embodiments the primer binding site further comprises restriction endonuclease sites which allow for the cleavage of the primer from the fully synthesized nucleic acid.

Enzymatic synthesis of nucleotides makes use of polymerases to add nucleotides to a growing nucleotide chain. Most polymerases function by forming a phosphodiester bond between an incoming nucleotide which has formed a base pair with the complimentary nucleotide in the template. This process requires the presence of a template that is complimentary to the sequence that is to be synthesized. However, according to the present invention a universal template may be used which allows for the synthesis of any sequence by adding one base at a time. Therefore, the universal template further comprises 10 to 200 universal bases, optionally at the 5′ end of the molecule. In particular embodiments the universal template may comprise 50 to 175, 50 to 150, 50 to 125, 50 to 100, 50 to 75, 75 to 200, 75 to 175, 75 to 150, 75 to 125, or 75 to 100 universal bases. A universal base includes bases that are able to transform their structure such as through keto-enol tautomerization so that they are able to base pair with all four naturally occurring nucleobases. Such bases may be used to form a universal template. One universal base is inosine which is capable of forming a base pair with adenine, cytosine, guanine and thymine. Another example of a universal base is Pyrimido[4,5-d]pyrimidine-2,4,5,7(1H,3H,6H,8H)-tetrone (PPT) available from Tokoyo Future Style Inc., Ibaraki, Japan. In some embodiments the universal template may further comprise at either or both the 3′ end and 5′end conventional bases (such as A, T. G and/or C) which may serve as primer binding sites, cleavage sites (such as type IIs restriction enzyme recognition and/or cleavage sites), terminal labeling for capture or other functions. In some embodiments, about 5% to about 10%, or about 5% to about 20%, or about 10% to 20%, or about 10% to 50%, or about 20% to 50% or about 30% to about 60% of the universal template may comprise conventional bases.

The nucleic acid molecule is synthesized using a polymerase to add 3′ protected nucleotides to extend the primer. The use of protected nucleotides allows for the addition of a single nucleotide thereby allowing for control of the sequence of the synthesized molecule. DNA polymerases which may be used include, but are not limited to, E. coli DNA polymerase, SEQUENASE 2.0™, T4 DNA polymerase or the Klenow fragment of DNA polymerase 1, T3, SP6 RNA polymerase, AMV, M-MLV, and/or Vent polymerase, as well as THERMOSEQUENASE™ (Amersham) or TAQUENASE™ (ScienTech, St Louis, Mo.).

Chemical groups that may be used to protect the 3′ position of the nucleotide are limited in that they should not interfere with the function of the polymerase and they must be easily converted to 3′-OH under mild conditions to allow for addition of the next oligonucleotide. Chemical groups which meet these criteria include 3′-O-allyl, 3′-O-methoxymethyl, 3′-O-nitrobenzyl, 3′-O-azidomethylene, and 3′-O-aminoalkoxyl compounds. In particular embodiments the protective group may be 3′-ONH₂. The 3′-ONH₂ may be converted to 3′-OH by exposure to sodium nitrite at a pH of about 5.5 for about 1 minute. The use of 3′-ONH₂ as a protective group and sodium nitrite as a deprotectant is described by Hutter et al., Nucleosides Nucleotides Nucleic Acids, 29(11), (2010). The use of UV light to remove the protective group 3′-O-(2-Nitrobenzyl)-dATP has been described by Metzker et al., Nucleic Acids Research, 22(20): 4259-4267 (1994). Using UV light as a deprotectant would allow the use of a single large surface with selective deprotection of synthesis spots with a directed beam of UV light. In such an embodiment only those spots where a nucleotide has been added would be subjected to the UV deprotection step for each round of synthesis.

In some embodiments, 3′ protective groups may be removed by the use of electrogenerated acid. In such embodiments the present invention for enzymatic synthesis may be part of a system for the synthesis of nucleic acids. Such a system would allow for the automated synthesis synthesis of nucleic acids using a completely aqueous based process. For example the present invention could be incorporated into the nucleic acid synthesis system described in WO2016094512. Replacement of conventional phosphoramidite chemistry for the addition of nucleotides with the present invention would allow the elimination of the use of organic solvents reducing the cost and environmental impact of the process.

As one skilled in the art would understand, many aspects of the invention are well suited for automation. Automated systems are often driven by software which may perform repetitive tasks, especially when integrated with hardware designed for micromanipulation of components and reagent flows. Thus, according to various embodiments described herein, methods of enzymatic synthesis of nucleic acids may be implemented with the use of a computing system. Further, according to various embodiments described herein, processor-executable instructions for synthesizing and optionally assembling nucleic acids are disclosed. Thus, some aspects the invention include a non-transitory computer-readable storage media encoded with instructions, executable by a processor, for generating synthesized nucleic acid molecules, the instructions comprising instructions for:

a) receiving from a user, the sequence of the nucleic acid to be synthesized,

b) adding to a solid support comprising a single stranded template comprising a primer binding site at the 3′ end and a universal template at the 5′ end a primer complimentary to the primer binding site,

c) adding a 3′ protected nucleotide and optionally a polymerase such that the protected nucleotide is added to the primer or the oligonucleotide extending from the primer and wherein the 3′ protected nucleotide is determined by the sequence received from the user,

d) removing the polymerase and unreacted protected nucleotide,

e) removing the protective group from the 3′ protected nucleotide; and

f) repeating steps (c)-(e) until synthesis of the oligonucleotide has been completed.

In particular embodiments, the solid support is the surface of a well or a tube or one or more beads which are in a well or tube. In these embodiments the reagents needed for each step are added to the well or tube and removed after completion of the step. In alternate embodiments where the solid support is a bead or similar particle then the bead or particle can be moved to different wells having the proper reagents for the next step in the synthesis.

In a further embodiment instructions may be provided for (g) combining the nucleic acid molecules generated in (e) to produce a pool;

(g) joining some or all of the nucleic acid molecules present in the pool formed in (f) to form a plurality of larger nucleic acid molecules;

(h) eliminating nucleic acid molecules which contain sequence errors from the plurality of larger nucleic acid molecules formed in (g) to produce an error corrected nucleic acid molecule pool.

Thus, in many instances, error correction will be desirable. Error correction can be achieved by any number of means. One method is by individually sequencing chemically synthesized nucleic acid molecules. Sequence-verified nucleic acid molecules can then be retrieved by various means. One way of selecting nucleic acid molecules of correct sequence is referred to as “laser catapulting” and relies on the use of high-speed laser pulses to eject selected clonal nucleic acid populations from a sequencing plate. This method is described, for example, in U.S. Patent Publication No. 2014/0155297 the disclosure of which is incorporated herein by reference.

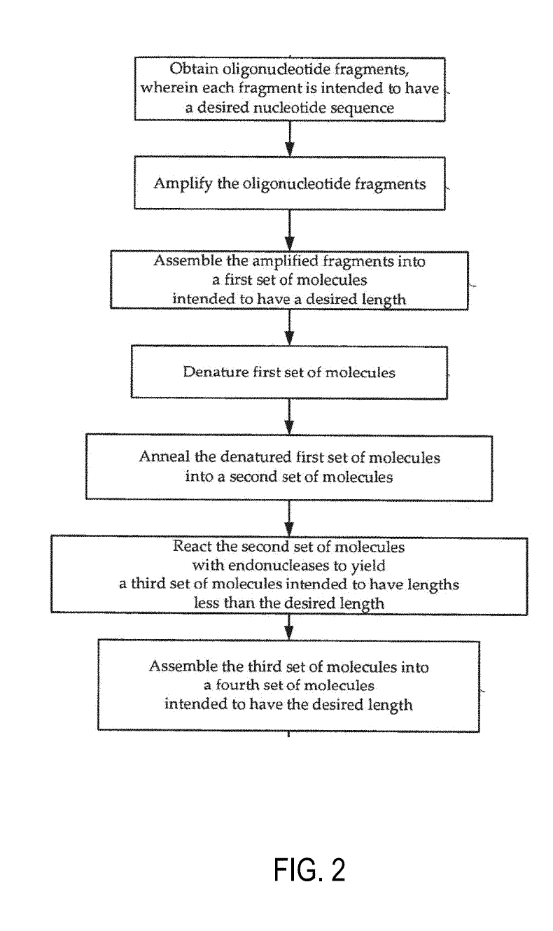

Another method of error correction is set out in FIG. 2. FIG. 2 is a flow chart of an exemplary process for synthesis of error-minimized nucleic acid molecules. In the first step, nucleic acid molecules of a length smaller than that of the full-length desired nucleotide sequence (i.e., “nucleic acid molecule fragments” of the full-length desired nucleotide sequence) are obtained. Each nucleic acid molecule is intended to have a desired nucleotide sequence that comprises a part of the full length desired nucleotide sequence. Each nucleic acid molecule may also be intended to have a desired nucleotide sequence that comprises an adapter primer for PCR amplification of the nucleic acid molecule, a tethering sequence for attachment of the nucleic acid molecule to a DNA microchip, or any other nucleotide sequence determined by any experimental purpose or other intention. The nucleic acid molecules may be obtained in any of one or more ways, for example, through synthesis, purchase, etc.

In the optional second step, the nucleic acid molecules are amplified to obtain more of each nucleic acid molecule. In many instances, however, sufficient numbers of nucleic acid molecules will be produced so that amplification is not necessary. When employed, amplification may be accomplished by any method known in the art, for example, by PCR, Rolling Circle Amplification (RCA), Loop Mediated Isothermal Amplification (LAMP), Nucleic Acid Sequence Based Amplification (NASBA), Strand Displacement Amplification (SDA), Ligase Chain Reaction (LCR), Self Sustained Sequence Replication (3SR), Recombinase Polymerase Amplification (RPA) or solid phase PCR reactions (SP-PCR) such as Bridge PCR etc. (see e.g. Fakruddin et al., J Pharm Bioallied Sci. 2013; 5(4): 245-252 for an overview of the various amplification techniques). Introduction of additional errors into the nucleotide sequences of any of the nucleic acid molecules may occur during amplification. In certain instances it may be favorable to avoid amplification following synthesis. The optional amplification step may be omitted where nucleic acid molecules have been produced at sufficient yield in the first step. This may be achieved by using improved compositions and methods of the invention such as e.g. optimized bead formats as described e.g. in WO2016094512, designed to allow synthesis of nucleic acid molecules at sufficient yield and quality.

In the third step, the optionally amplified nucleic acid molecules are assembled into a first set of molecules intended to have a desired length, which may be the intended full length of the desired nucleotide sequence. Assembly of amplified nucleic acid molecules into full-length molecules may be accomplished in any way, for example, by using a PCR-based method.

In the fourth step, the first set of full-length molecules is denatured. Denaturation renders single-stranded molecules from double-stranded molecules. Denaturation may be accomplished by any means. In some embodiments, denaturation is accomplished by heating the molecules.

In the fifth step, the denatured molecules are annealed. Annealing renders a second set of full-length, double-stranded molecules from single-stranded molecules. Annealing may be accomplished by any means. In some embodiments, annealing is accomplished by cooling the molecules. Some of the annealed molecules may contain one or more mismatches indicating sites of sequence error.

In the sixth step, the second set of full-length molecules are reacted with one or more mismatch cleaving endonucleases to yield a third set of molecules intended to have lengths less than the length of the complete desired gene sequence. The endonucleases cut one or more of the molecules in the second set into shorter molecules. The cuts may be accomplished by any means. Cuts at the sites of any nucleotide sequence errors are particularly desirable, in that assembly of pieces of one or more molecules that have been cut at error sites offers the possibility of removal of the cut errors in the final step of the process. In an exemplary embodiment, the molecules are cut with T7 endonuclease I, E. coli endonuclease V, and Mung Bean endonuclease in the presence of manganese. In this embodiment, the endonucleases are intended to introduce cuts in the molecules at the sites of any sequence errors, as well as at random sites where there is no sequence error. In another exemplary embodiment, the molecules are cut only with one endonuclease (such as T7 endonuclease I or another endonuclease of similar functionality).

In the seventh step, the third set of molecules is assembled into a fourth set of molecules, whose length is intended to be the full length of the desired nucleotide sequence. Because of the late-stage error correction enabled by the provided method, the set of molecules is expected to have many fewer nucleotide sequence errors than can be provided by methods in the prior art. Optionally, steps four to seven may be repeated one or several times to further increase the efficiency of error reduction.

The process set out above and in FIG. 2 is also set out in U.S. Pat. No. 7,704,690, the disclosure of which is incorporated herein by reference. Furthermore, the process described above may be encoded onto a computer-readable medium as processor-executable instructions.

Another process for effectuating error correction in chemically synthesized nucleic acid molecules is by a commercial process referred to as ERRASE™ (Novici Biotech). Error correction methods and reagent suitable for use in error correction processes are set out in U.S. Pat. Nos. 7,838,210 and 7,833,759, U.S. Patent Publication No. 2008/0145913 A1 (mismatch endonucleases), and PCT Publication WO 2011/102802 A1, the disclosures of which are incorporated herein by reference.

Exemplary mismatch binding and/or cleaving enzymes include endonuclease VII (encoded by the T4 gene 49), RES I endonuclease, CEL I endonuclease, and SP endonuclease or an endonuclease containing enzyme complex. For example, the MutHLS complex constitutes a bacterial mismatch repair system, wherein MutS has mismatch detection and mismatch binding activity, MutH has nuclease activity and MutL directs MutH to MutS-bound mismatch sites. The skilled person will recognize that other methods of error correction may be practiced in certain embodiments of the invention such as those reviewed in Ma et al., Trends in Biotechnology, 30(3): 147-154 (2012), or those described, for example, in U.S. Patent Publication Nos. 2006/0127920 AA, 2007/0231805 AA, 2010/0216648 A1, 2011/0124049 A1 or U.S. Pat. No. 7,820,412, the disclosures of which are incorporated herein by reference.

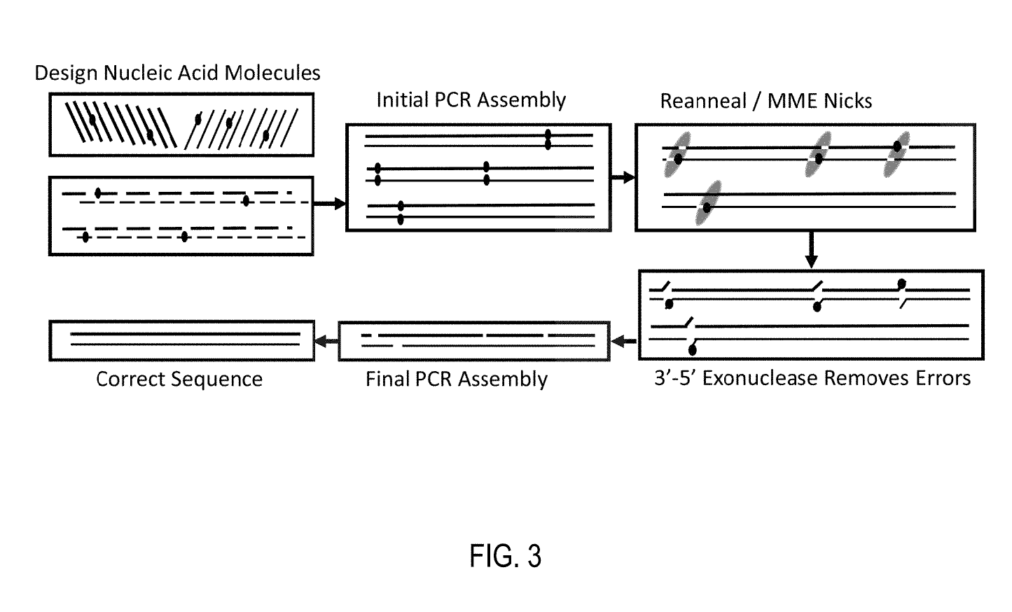

Another schematic of an error correction method is shown in FIG. 3.

Synthetically generated nucleic acid molecules typically have error rates of about 1 base in 300-500 bases. As noted above in many instances, conditions can be adjusted so that synthesis errors are substantially lower than 1 base in 300-500 bases. Further, in many instances, greater than 80% of errors are single base frameshift deletions and insertions. Also, less than 2% of errors result from the action of polymerases when high fidelity PCR amplification is employed. In many instances, mismatch endonuclease (MME) correction will be performed using fixed protein:DNA ratio.

One error correction methods involves the following steps. The first step is to denature DNA contained in a reaction buffer (e.g., 200 mM Tris-HCl (pH 8.3), 250 mM KCl, 100 mM MgCl₂, 5 mM NAD⁺, and 0.1% TRITON® X-100) at 98° C. for 2 minutes, followed by cooling to 4° C. for 5 minutes, then warming the solution to 37° C. for 5 minutes, followed by storage at 4° C. At a later time, T7endonuclease I and DNA ligase are added and the solution is incubated at 37° C. for I hour. The reaction is stopped by the addition EDTA. A similar process is set out in Huang et al., Electrophoresis 33:788-796 (2012).

Another method for removal of errors from chemically synthesized nucleic acid molecules is by selection of nucleic acid molecules having correct nucleotide sequences. This may be done by the selection of a single nucleic acid molecule for amplification, then sequencing of the amplification products to determine if any errors are present. Thus, the invention also includes selection methods for the reduction of sequence errors. Methods for amplifying and sequence verifying nucleic acid molecules are set out in U.S. Pat. No. 8,173,368, the disclosure of which is incorporated herein by reference. Similar methods are set out in Matzas et al., Nature Biotechnology, 28:1291-1294 (2010). Selection of sequence-verified nucleic acid molecules can be accomplished by various means including methods using laser pulses as described elsewhere herein.

The invention also includes compositions and methods for the isolation of nucleic acid molecules that have a desired nucleotide sequence present in a population of nucleic acid molecules that do not have the desired sequence (e.g., have “errors”). This may be done using methods in which nucleic acid molecules containing errors are physically separated from nucleic acid molecules that have the “correct” nucleotide sequence. The invention further includes compositions and methods by which nucleic acid molecules are not subjected to in vitro amplification steps, or other steps, that may introduce errors. Thus, as part of, for example, the error correction process, nucleic acid molecules having correct sequences may be physically separated from those that do not have the correct sequence. One means by which to do this involves the use of agents that bind nucleic acid molecules that contain mismatches.

As an example, a protein that has been shown to bind double-stranded nucleic acid molecules containing mismatches is E. coli MutS (Wagner et al., Nucleic Acids Res., 23:3944-3948 (1995)). Wan et al., Nucleic Acids Res., 42:e102 (2014) demonstrated that chemically synthesized nucleic acid molecules containing errors can be retained on a MutS-immobilized cellulose column with nucleic acid molecules not containing errors not being so retained.

The invention thus includes methods, as well as associated compositions, in which nucleic acid molecules are denatured, followed by reannealing, followed by the separation of reannealed nucleic acid molecules containing mismatches. In some aspects, the mismatch binding protein used is MutS (e.g., E. coli MutS).

Further, mixtures of mismatch repair binding proteins may be used in the practice of the invention. It has been found that different mismatch repair binding proteins have different activities with respect to the types of mismatches they bind to. For example, Thermus aquaticus MutS has been shown to effectively remove insertion/deletion errors but is less effective in removing substitution errors than E. coli MutS. Further, a combination the two MutS homologs was shown to further improve the efficiency of the error correction with respect to the removal of both substitution and insertion/deletion errors, and also reduced the influence of biased binding. The invention thus includes mixtures of two or more (e.g., from about two to about ten, from about three to about ten, from about four to about ten, from about two to about five, from about three to about five, from about four to about six, from about three to about seven, etc.) mismatch repair binding proteins.

The invention further includes the use of multiple rounds (e.g., from about two to about ten, from about three to about ten, from about four to about ten, from about two to about five, from about three to about five, from about four to about six, from about three to about seven, etc.) of error correction using mismatch repair binding proteins. One or more of these rounds of error correction may employ the use of two or more mismatch repair binding proteins. Alternatively, a single mismatch repair binding protein may be used in a first round of error correction whereas the same or another mismatch binding protein may be used in a second round of error correction.

Methods according to this aspect of the invention may include the following steps: (a) providing a mixture of nucleic acid molecules synthesized to have the same nucleotide sequence, (b) separating nucleic acid molecules in the mixture such that amplification results in progeny nucleic acid molecules being derived from a single starting nucleic acid molecule, (c) sequencing more than one amplified nucleic acid molecule generated in step (b), and (d) identifying at least one individual nucleic acid with the desired sequence from the nucleic acid molecules sequenced in step (c). The nucleic acid molecule identified in step (d) may then be used as one nucleic acid molecule in an assembly process, as described elsewhere herein.

In some circumstances it may be useful to amplify the synthesized nucleic acid strand. Amplification can be achieved by any methods known in the art and/or disclosed herein for amplifying nucleic acid molecules. When polymerase chain reaction (PCR) amplification is used, conditions can include the presence of ribonucleotide and/or deoxyribo-nucleotide di-, tri-, tetra-, penta- and/or higher order phosphates; primers for PCR amplification for at least one nucleic acid and its corresponding competitive template; and at least one polymerization-inducing agent, such as reverse transcriptase, RNA polymerase and/or DNA polymerase. Examples of DNA polymerases include, but are not limited to, E. coli DNA polymerase, SEQUENASE 2.0™, T4 DNA polymerase or the Klenow fragment of DNA polymerase 1, T3, SP6 RNA polymerase, AMV, M-MLV, and/or Vent polymerase, as well as THERMOSEQUENASE™ (Amersham) or TAQUENASE™ (ScienTech, St Louis, Mo.). Further examples include thermostable polymerases isolated from Thermus aquaticus, Thermus thermophilus, Pyrococcus woesei, Pyrococcus furiosus, Thermococcus litoralis, and Thermotoga maritima. The polymerization-inducing agent and nucleotides may be present in a suitable buffer, which may include constituents which are co-factors or which affect conditions such as pH and the like at various suitable temperatures. PCR primers used are preferably single stranded, but double-, triple- and/or higher order stranded nucleotide molecules can be practiced with the present invention. As used herein “amplified product” can refer to any nucleic acid synthesized at least partly by base-complementary incorporation using another nucleic acid as template. An amplified product may also be referred to an amplicon and/or amplimer herein. Amplification may be carried out for a number of cycles of PCR, e.g., at least about 10, at least about 20, at least about 30, at least about 35, at least about 40, or at least about 50 cycles in some embodiments.

In some embodiments, multiple synthesized nucleic acid molecules may be combined to form a larger nucleic acid molecule. A commercially available product for the assembly of nucleic acid molecules in yeast cells is the GENEART® High-Order Genetic Assembly Systems (Thermo Fisher Scientific, Cat. No. A13286). This is a kit for the simultaneous and seamless assembly of up to 10 DNA fragments, totaling up to 110 kilobases in length, into vectors. The system uses the ability of yeast to take up and recombine DNA fragments with high efficiency. This greatly reduces the in vitro handling of DNA and eliminates the need for enzymatic treatments, such as restriction and ligation, while allowing for precise fusions of DNA sequences. The kit contains materials for the transformation and purification from yeast, including yeast selective media, and competent E. coli for plasmid amplification of correct clones.

### EXAMPLE

A template 48mer oligonucleotide 5′-CGGTACCTGCATGCCGAC-XXXXXXXXXXXX-CAGCTAGACTAGAGCTCG-Biotin-3′ (SEQ ID NO:1) is synthesized using standard phosphoamidite chemistry. The constant 5′ region contains a KpnI cleavage site, the 3′ region contains a SacI cleavage site. A biotin molecule is attached during synthesis 5′ of this molecule, ACGT are standard desoxyribonucleic acid, X is the universal base Pyrimido[4,5-d]pyrimidine-2,4,5,7(1H,3H,6H,8H)-tetrone (PPT) (Tokyo Future Style Inc). Because PPT is only available as a PPT-2-TBDMS amidite building block, leaving a 2′ OH after deprotection, this part of the molecule is considered RNA. The center part of the oligonucleotide contains 12 consecutive incorporations of PPT. This molecule is further referred to as “template oligo” (Ot). A primer 18mer oligonucleotide 5′-CGAGCTCTAGTCTAGCTG-3′ (SEQ ID NO:2) is synthesized using standard phosphoamidite chemistry and is further referred to as “primer oligo” (Op). Op is the reverse complement of the 3′ end of Ot.

100 pmol of Ot is incubated with 5 ul of Dynabeads M-280 Streptavidin (Thermo Fisher Scientific) in a total volume of 50 ul PBS at 37° C. for 30 min. Because the initial Dynabeads concentration is 10 mg/ml a total of 0.05 mg of Dynabeads is used and is expected to bind 10 pmol of biotinylated ssDNA according to the manufacturer. After 30 min, beads are sedimented with a magnet, supernatant is discarded and beads are washed twice with 100 ul PBS and resuspended in a final volume of 50 ul PBS. 100 pmol of Op is then added and the mixture heated to 75° C. for 5 min, then slowly cooled to room temperature over a course of 5 min to allow Op to hybridize to the 3′ end of Ot. Beads are then washed twice with 100 ul of PBS, once with 100 ul M-MLV RT buffer (50 mM Tris-HCl (pH 8.3), 75 mM KCl, 3 mM MgCl₂, 10 mM DTT) and resuspended in a final volume of 20 ul M-MLV RT buffer, supplemented with 0.1% hydroxylamine.

To enzymatically elongate Op with the first nucleotide, cytosine, we add to this reaction a volume of 5 ul containing 0.05 ul (10 U) M-MLV RT (Thermo Fisher Scientific) and 100 uM 3′-O-alkyl hydroxylamine CTP in M-MLV RT buffer. The reaction is incubated at 37° C. for 1 min, supernatant is discarded and beads are resuspended in cleavage buffer (0.7 M NaNO₂, 1 M NaOAc (pH 5.5)) and incubated at 37° C. for 1 min. Beads are then washed twice with 50 ul M-MLV RT buffer, resuspended in a final volume of 20 ul M-MLV RT buffer, supplemented with 0.1% hydroxylamine and the next elongation step with adenine is performed by adding a volume of 5 ul containing 0.05 ul (10 U) M-MLV RT (Thermo Fisher Scientific) and 100 uM 3′-O-alkyl hydroxylamine ATP in M-MLV RT buffer. These consecutive steps are iteratively repeated another 10 times to sequentially elongate Op along the 12 universal PPT template positions. The full sequential order of elongation is: CATGGATCCTGA (SEQ ID NO:3) (contains BamHI cleavage site).

After cleavage of the last A, beads are washed twice in 50 ul Klenow buffer (50 mM NaCl, 10 mM Tris-HCl, 10 mM MgCl₂, 1 mM DTI (pH 7.9)), resuspended in 20 ul Klenow buffer including 1 U Klenow Fragment (3′→5′ exo-) and 100 μM dNTPs and incubated at 37° C. for 5 min to elongate Op to the end according to the fixed 5′ end of Ot.

To obtain the elongated ssDNA(Oe) derived from elongation of Op the supernatant of the Klenow reaction is discarded and beads are resuspended in 100 ul SSC (0.15 M NaCl, 0.015 M sodium citrate (pH 7.0)) and heated to 95° C. for 5 min. Beads are quickly sedimented with a magnet and Oe ssDNA containing supernatant is transferred into a new tube.

Oe (0.1 ul) is amplified with primer “oligo forward” (Of) 5′-CGAGCTCTAGTCTAGCTG-3′ (SEQ ID NO:4) and “oligo reverse” (Or) 5′-CGGTACCTGCATGCCGAC-3′ (SEQ ID NO:5) in a standard PCR under the following conditions: 25× (30 sec at 95° C., 30 sec at 55° C., 30 sec at 72° C.). The PCR product is cleaved with BamHI at 370 for 30 min. Cleaved and uncleaved product is analyzed on 3% agarose. The amount of DNA amenable to BamHI cleavage is estimated at 75% based on densitometric analysis of DNA bands.

Another aliquot of the PCR product is cleaved with restriction enzymes KpnI and SacI, ligated into a cloning vector and transformed into E. coli. Colonies are analyzed by colony PCR and Sanger sequencing of colony PCR products. Analysis of an alignment from a peer group of 96 colonies shows incorporation of the correct nucleotide at 95% per position. The majority of errors in the 12 bp region are single base pair deletions. Overall 53 of 96 analyzed clones contained the correct sequence: CATGGATCCTGA (SEQ ID NO:6). In other examples, incorporation of the correct nucleotide is found at 90% per position.

It will be appreciated that, for clarity purposes, the above description has described embodiments of the invention with reference to different functional units and processors. However, it will be apparent that any suitable distribution of functionality between different functional units, processors or domains may be used without detracting from the invention. For example, functionality illustrated to be performed by separate processors or controllers may be performed by the same processor or controller. Hence, references to specific functional units are only to be seen as references to suitable means for providing the described functionality, rather than indicative of a strict logical or physical structure or organization.

All publications, patents and patent applications mentioned in this Specification are indicative of the level of skill of those of ordinary skill in the art and are herein incorporated by reference to the same extent as if each individual publication, patent, or patent applications was specifically and individually indicated to be incorporated by reference.

The invention being thus described, one skilled in the art would recognize that the invention may be varied in many ways. Such variations are not to be regarded as a departure from the spirit and scope of the invention, and all such modifications as would be obvious to one of ordinary skill in the art are intended to be included within the scope of the following claims.

The invention is further described by the following clauses:

Clause 1. A method for the synthesis of an oligonucleotide comprising: a) providing on a solid support a single or double stranded template comprising a primer binding site at the 3′ end and a universal template, b) adding a primer complimentary to the primer binding site, c) adding a polymerase, d) adding a 3′ protected nucleotide such that the protected nucleotide is added to the primer or the oligonucleotide extending from the primer, e) removing the unreacted protected nucleotide, f) removing the protective group from the 3′ protected nucleotide; and g) repeating steps (d)-(f) and optionally step (c) until synthesis of the oligonucleotide has been completed.

Clause 2. A method for the synthesis of an oligonucleotide comprising: a) providing on a solid support a single or double stranded template comprising a primer binding site at the 3′ end and a universal template at the 5′ end, b) adding a primer complimentary to the primer binding site, c) adding a polymerase and a 3′ protected nucleotide such that the protected nucleotide is added to the primer or the oligonucleotide extending from the primer, d) removing the unreacted protected nucleotide, e) removing the protective group from the 3′ protected nucleotide; and f) repeating steps (c)-(e) until synthesis of the oligonucleotide has been completed.

Clause 3. A method for the synthesis of an oligonucleotide comprising: a) providing on a solid support a single or double stranded template comprising a primer binding site at the 3′ end and a universal template at the 5′ end, b) adding a primer complimentary to the primer binding site, c) adding a 3′ protected nucleotide such that the protected nucleotide is added to the primer or the oligonucleotide extending from the primer in the presence of a polymerase, d) removing the unreacted protected nucleotide, e) removing the protective group from the 3′ protected nucleotide; and f) repeating steps (c)-(e) until synthesis of the oligonucleotide has been completed.

Clause 4. The method of clause 2 or 3, wherein step d) further comprises removing the polymerase.

Clause 5. The method of any preceding clause, wherein the primer binding site is from 10 to 25 nucleotides in length.

Clause 6. The method of any preceding clause, wherein the universal template is from 10 to 200 nucleotides in length.

Clause 7. The method of any preceding clause, wherein the universal template comprises a universal base.

Clause 8. The method of clause 7, wherein the universal base is selected from the group consisting of inosine and PPT.

Clause 9. The method of any preceding clause, wherein the protective group of the 3′ protected molecule is selected from the group consisting of 3′-O-allyl, 3′-O-methoxymethyl, 3′-O-nitrobenzyl, 3′-O-azidomethylene, and 3′-O-aminoalkoxyl.

Clause 10. The method of any of clauses 3-9, wherein the polymerase is added together with the 3′ protected nucleotide in step (c).

Clause 11. A non-transitory computer-readable storage media encoded with instructions, executable by a processor, for generating synthesized nucleic acid molecules, the instructions comprising instructions for: a) adding to a solid support comprising a single or double stranded template comprising a primer binding site at the 3′ end and a universal template at the 5′ end a primer complimentary to the primer binding site, b) adding a polymerase and a 3′ protected nucleotide such that the protected nucleotide is added to the primer or the oligonucleotide extending from the primer, c) removing the unreacted protected nucleotide, d) removing the protective group from the 3′ protected nucleotide; and e) repeating steps (b)-(d) until synthesis of the oligonucleotide has been completed.

Clause 12. The non-transitory computer-readable storage media of clause 10, wherein step c) further comprises removing the polymerase.

Clause 13. The non-transitory computer-readable storage media of clause 11, further comprising providing instructions for: (f) combining the nucleic acid molecules generated in (e) to produce a pool; (g) joining some or all of the nucleic acid molecules present in the pool formed in (f) to form a plurality of larger nucleic acid molecules.

Clause 14. The non-transitory computer-readable storage media of clause 13, further comprising eliminating nucleic acid molecules which contain sequence errors from the plurality of larger nucleic acid molecules formed in (g) to produce an error corrected nucleic acid molecule pool.

Clause 15. The non-transitory computer-readable storage media of clause 11, further comprising instructions for receiving from a user the nucleotide sequence to be synthesized.

Clause 16. The non-transitory computer-readable storage media of clause 15, wherein the user uses a keyboard or similar input device to enter the sequence to be synthesized.

Clause 17. The non-transitory computer-readable storage media of clause 15, wherein the user provides a FASTA file containing the sequence to be synthesized.

What is claimed is:

1. A method for the synthesis of an oligonucleotide comprising:
a) providing on a solid support a single or double stranded template comprising a primer binding site at the 3′ end and a universal template,
b) adding a primer complimentary to the primer binding site,
c) adding a polymerase,
d) adding a 3′ protected nucleotide such that the protected nucleotide is added to the primer or the oligonucleotide extending from the primer,
e) removing the unreacted protected nucleotide,
f) removing the protective group from the 3′ protected nucleotide; and
g) repeating steps (d)-(f) and optionally step (c) until synthesis of the oligonucleotide has been completed.

2. The method of claim 1, wherein step e) further comprises removing the polymerase.

3. The method of claim 1, wherein the primer binding site is from 10 to 25 nucleotides in length.

4. The method of claim 1, wherein the universal template is from 10 to 200 nucleotides in length.

5. The method of claim 1, wherein the universal template comprises a universal base.

6. The method of claim 5, wherein the universal base is selected from the group consisting of inosine and PPT.

7. The method of claim 1, wherein the protective group of the 3′ protected molecule is selected from the group consisting of 3′-O-allyl, 3′-O-methoxymethyl, 3′-O-nitrobenzyl, 3′-O-azidomethylene, and 3′-O-aminoalkoxyl.

8. A non-transitory computer-readable storage media encoded with instructions, executable by a processor, for generating synthesized nucleic acid molecules, the instructions comprising instructions for:
a) adding to a solid support comprising a single or double stranded template comprising a primer binding site at the 3′ end and a universal template and a primer complimentary to the primer binding site,
b) adding a polymerase,
c) adding a 3′ protected nucleotide such that the protected nucleotide is added to the primer or the oligonucleotide extending from the primer,
d) removing the unreacted protected nucleotide,
e) removing the protective group from the 3′ protected nucleotide; and
f) repeating steps (c)-(e) and optionally step (b) until synthesis of the oligonucleotide has been completed.

9. The non-transitory computer-readable storage media of claim 8, wherein step d) further comprises removing the polymerase.

10. The non-transitory computer-readable storage media of claim 8, further comprising providing instructions for:
(f) combining the nucleic acid molecules generated in (e) to produce a pool;
(g) joining some or all of the nucleic acid molecules present in the pool formed in
(f) to form a plurality of larger nucleic acid molecules.

11. The non-transitory computer-readable storage media of claim 10, further comprising eliminating nucleic acid molecules which contain sequence errors from the plurality of larger nucleic acid molecules formed in (g) to produce an error corrected nucleic acid molecule pool.

12. The non-transitory computer-readable storage media of claim 8, further comprising instructions for receiving from a user the nucleotide sequence to be synthesized.

13. The non-transitory computer-readable storage media of claim 12, wherein the user uses a keyboard or similar input device to enter the sequence to be synthesized.

14. The non-transitory computer-readable storage media of claim 12, wherein the user provides a FASTA file containing the sequence to be synthesized.
